(19) United States
(12) Patent Application Publication
(10) Pub. No.: US 2017/0166857 A1
(43) Pub. Date: Jun. 15, 2017

(54) Mammalian Cell In Vitro Topological Neuron Network

(71) Applicant: Robert John Petcavich, The Woodlands, TX (US)

(72) Inventor: Robert John Petcavich, The Woodlands, TX (US)

(21) Appl. No.: 15/374,961

(22) Filed: Dec. 9, 2016

Related U.S. Application Data
(60) Provisional application No. 62/265,399, filed on Dec. 9, 2015.

(51) Int. Cl. C12M 1/34; C12M 1/32

(52) U.S. Cl. CPC C12M 41/46; C12M 23/12

(57) ABSTRACT

A method of creating a three-dimensional surface topography network for the containment and growth of mammalian neuron cells for high throughput screening of potential biologically active drug compounds is provided. In the present disclosure a n×n array of wells is created on a carrier substrate that contains both wells and interconnected channels between wells to facilitate, in one embodiment, axon growth between neuron cells and subsequently the creation of a living interactive neuron network in vitro that emulates in vivo neuron behavior.

### CROSS-REFERENCE TO RELATED APPLICATIONS

This application claims the benefit of the filing date of U.S. application Ser. No. 62/265,399, filed on Dec. 9, 2015, the disclosure of which is incorporated by reference herein.

### TECHNICAL FIELD

The present disclosure is directed to a method of creating a physical and logical topological network for stem cells in vitro. In one embodiment, a substrate is provided which comprises a pattern to facilitate the growth of neurons and axons to create a living biologically functional network that can be monitored electrically and optically to determine the effect of biologically active compounds on neuron signaling and communications.

### BACKGROUND

Network topology generally refers to the arrangement of the various elements, links, nodes, of a computer network. Essentially, it is the topological structure of a network and may be depicted physically or logically. Physical topology is the placement of the various components of a network, including device location and cable installation, while logical topology illustrates how data flows within a network, regardless of its physical design. Distances between nodes, physical interconnections, transmission rates, or signal types may differ between two networks, yet their topologies may be identical. An example is a local area network (LAN): Any given node in the LAN has one or more physical links to other devices in the network; graphically mapping these links results in a geometric shape that can be used to describe the physical topology of the network. Conversely, mapping the data flow between the components determines the logical topology of the network.

The cabling layout used to link devices is the physical topology of the network. This refers to the layout of cabling, the locations of nodes, and the interconnections between the nodes and the cabling. The physical topology of a network is determined by the capabilities of the network access devices and media, the level of control or fault tolerance desired, and the cost associated with cabling or communications circuits. The logical topology in contrast, is the way that the signals act on the network media, or the way that the data passes through the network from one device to the next without regard to the physical interconnection of the devices. A network's logical topology is not necessarily the same as its physical topology.

The logical classification of network topologies generally follows the same classifications as those in the physical classifications of network topologies but describes the path that the data takes between nodes being used as opposed to the actual physical connections between nodes. The logical topologies are generally determined by network protocols as opposed to being determined by the physical layout of cables, wires, and network devices or by the flow of the electrical signals, although in many cases the paths that the electrical signals take between nodes may closely match the logical flow of data, hence the convention of using the terms logical topology and signal topology interchangeably.

The study of network topology recognizes eight basic topologies: point-to-point, bus, star, ring or circular, mesh, tree, hybrid, or daisy chain. FIG. 1 shows a diagram of various examples of network topologies.

The simplest topology with a dedicated link between two endpoints (point-to-point). Switched point-to-point topologies are the basic model of conventional telephony. The value of a permanent point-to-point network is unimpeded communications between the two endpoints. The value of an on-demand point-to-point connection is proportional to the number of potential pairs of subscribers and has been expressed as Metcalfe's Law. Easiest to understand of the variations of point-to-point topology is a point-to-point communications channel that appears, to the user, to be permanently associated with the two endpoints. A children's tin can telephone is one example of a physical dedicated channel.

In local area networks where bus topology is used, each node is connected to a single cable, by the help of interface connectors. This central cable is the backbone of the network and is known as the bus (thus the name). A signal from the source travels in both directions to all machines connected on the bus cable until it finds the intended recipient. If the machine address does not match the intended address for the data, the machine ignores the data. Alternatively, if the data matches the machine address, the data is accepted. Because the bus topology consists of only one wire, it is rather inexpensive to implement when compared to other topologies. However, the low cost of implementing the technology is offset by the high cost of managing the network. Additionally, because only one cable is utilized, it can be the single point of failure.

The type of network topology in which all of the nodes of the network are connected to a common transmission medium which has exactly two endpoints (this is the ‘bus’, which is also commonly referred to as the backbone, or trunk)—all data that is transmitted between nodes in the network is transmitted over this common transmission medium and is able to be received by all nodes in the network simultaneously.

In local area networks with a star topology, each network host is connected to a central hub with a point-to-point connection. So it can be said that every computer is indirectly connected to every other node with the help of the hub. In Star topology every node (computer workstation or any other peripheral) is connected to a central node called hub, router or switch. The switch is the server and the peripherals are the clients. The network does not necessarily have to resemble a star to be classified as a star network, but all of the nodes on the network must be connected to one central device. All traffic that traverses the network passes through the central hub. The hub acts as a signal repeater. The star topology is considered the easiest topology to design and implement. An advantage of the star topology is the simplicity of adding additional nodes. The primary disadvantage of the star topology is that the hub represents a single point of failure.

A network topology is set up in a circular fashion in such a way that they make a closed loop. This way data travels around the ring in one direction and each device on the ring acts as a repeater to keep the signal strong as it travels. Each device incorporates a receiver for the incoming signal and a transmitter to send the data on to the next device in the ring. The network is dependent on the ability of the signal to travel around the ring. When a device sends data, it must travel through each device on the ring until it reaches its destination. Every node is a critical link. In a ring topology, there is no server computer present; all nodes work as a server and repeat the signal. The disadvantage of this topology is that if one node stops working, the entire network is affected or stops working.

The value of fully meshed networks is proportional to the exponent of the number of subscribers, assuming that communicating groups of any two endpoints, up to and including all the endpoints, is approximated by Reed's Law.

A fully connected network is a communication network in which each of the nodes is connected to each other (fully connected mesh network). In graph theory it known as a complete graph. A fully connected network doesn't need to use switching or broadcasting. However, its major disadvantage is that the number of connections grows quadratically with the number of nodes, as per the formula:

$$c = \frac{n ( n - 1 )}{2} .$$

And so it is extremely impractical for large networks. A two-node network is technically a fully connected network. The human brain behaves like a fully connected network with trillions of connections that can be static or dynamic between all the neurons that give the brain network plasticity, which is great for learning, and processing new information.

A tree topology is essentially a combination of bus topology and star topology. The nodes of bus topology are replaced with standalone star topology networks. This results in both disadvantages of bus topology and advantages of star topology. For example, if the connection between two groups of networks is broken down due to breaking of the connection on the central linear core, then those two groups cannot communicate, much like nodes of a bus topology. However, the star topology nodes will effectively communicate with each other.

Hybrid networks use a combination of any two or more topologies, in such a way that the resulting network does not exhibit one of the standard topologies (e.g., bus, star, ring, etc.). For example, a tree network connected to a tree network is still a tree network topology. A hybrid topology is always produced when two different basic network topologies are connected. Two common examples for Hybrid network are: star ring network and star bus network. A Star ring network consists of two or more star topologies connected using a centralized hub. A Star Bus network consists of two or more star topologies connected using a bus trunk (the bus trunk serves as the network's backbone).

### SUMMARY

The human brain is a very complex network of individual neurons that are organized like one or more or all of the aforementioned networks. The present disclosure provides for a topological network of neurons on a carrier substrate in vitro that are interconnected by axons to study the behavior of signaling and communications between neurons when exposed to biologically active compounds. The present disclosure provides a platform to observe and measure both the physical and logical topology of a neuron network in vitro.

In one embodiment, a biocompatible substrate comprising a network of wells for culturing cells in vitro and channels connecting the wells is provided. In one embodiment, the substrate has two or more wells including media and connected by channels configured form a network between the two or more wells and to facilitate intercellular connections. In one embodiment, at least one of the wells has neurons, or stem or progenitor cells capable of differentiating into neural cells. In one embodiment, the network is a ring, mesh, star, line, tree or bus topology, e.g., a star or tree topology. In one embodiment, the substrate comprises glass or a synthetic polymer. In one embodiment, the substrate comprises a natural polymer. In one embodiment, the substrate comprises an electrode array. In one embodiment, the substrate is formed by liquid casting, injecting molding, thermal and/or UV micro embossing, micro machining, thermoforming, and/or high pressure stamping. In one embodiment, at least one well includes neuromuscular, cardiac, liver, kidney, pancreas, or skin cells. In one embodiment, at least one well includes motor neurons. In one embodiment, at least one well includes non-motor neurons. In one embodiment, the non-motor neurons comprise cortical neurons, hippocampal neurons or dorsal root neurons. In one embodiment, adjacent wells have different cell types. In one embodiment, at least one channel has a diameter of about 5 to about 250 microns. In one embodiment, at least one channel has a diameter of about 5 to about 25 microns. In one embodiment, at least one channel has a diameter of about 10 to about 20 microns. In one embodiment, at least one channel has a length of about 5 microns to about 1 millimeter. In one embodiment, at least one channel has a length of about 5 to about 25 microns. In one embodiment, at least one channel has a length of about 10 to about 22 microns.

Also provided is a method of making a biocompatible substrate having a network of wells for culturing cells in vitro and channels connecting the wells. In one embodiment, a multi-well plate is provided, wherein the diameter of the wells is about 10 microns to about 25000 microns in diameter. One or more channels is fabricated between one or more wells, wherein the width of the channels is about 2 microns to about 250 microns, thereby forming a network of interconnected wells. In one embodiment, a plurality of the channels has a width of about 5 to 25 microns. In one embodiment, a plurality of the channels has a length of about 5 to 25 microns. In one embodiment, the network is a ring, mesh, star, line, tree or bus topology. In one embodiment, the substrate comprises glass or a synthetic polymer. In one embodiment, the substrate comprises an electrode array.

The substrate may be employed, in one embodiment, to monitor cellular activity. For example, media in the wells of a substrate are contacted with one or more compounds, and the activity of the cells in the wells is monitored after contact. In one embodiment, electrical activity is monitored. In one embodiment, at least two wells are contacted with a different compound. In one embodiment, the media in each well is the same. In one embodiment, at least one well includes neuromuscular, cardiac, liver, kidney, pancreas, or skin cells. In one embodiment, the cells in the wells are the same type of cell. In one embodiment, at least one well includes motor neurons. In one embodiment, at least one well includes non-motor neurons. In one embodiment, the non-motor neurons comprise cortical neurons, hippocampal neurons or dorsal root neurons. In one embodiment, adjacent wells have different cell types. In one embodiment, one or more of the channels comprise axons.

### BRIEF DESCRIPTION OF THE DRAWINGS

FIG. 1 shows the various network configurations that can be fabricated in the present disclosure on a carrier substrate.

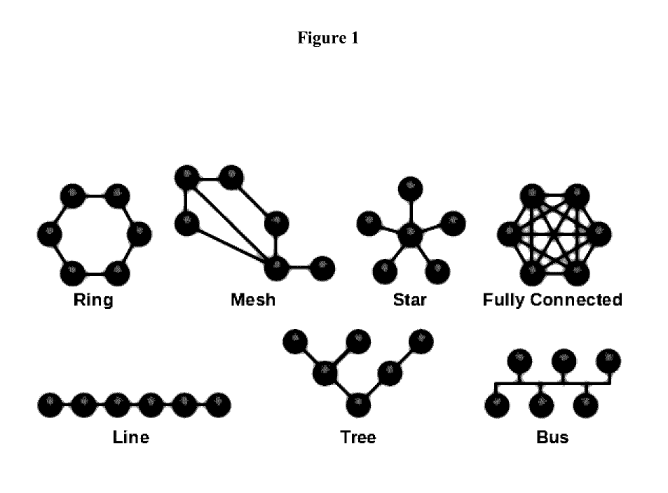

FIG. 2 shows a 3 by 3 array of wells and well interconnection channels to facilitate the growth of axons between neurons using the present disclosure.

### DETAILED DESCRIPTION

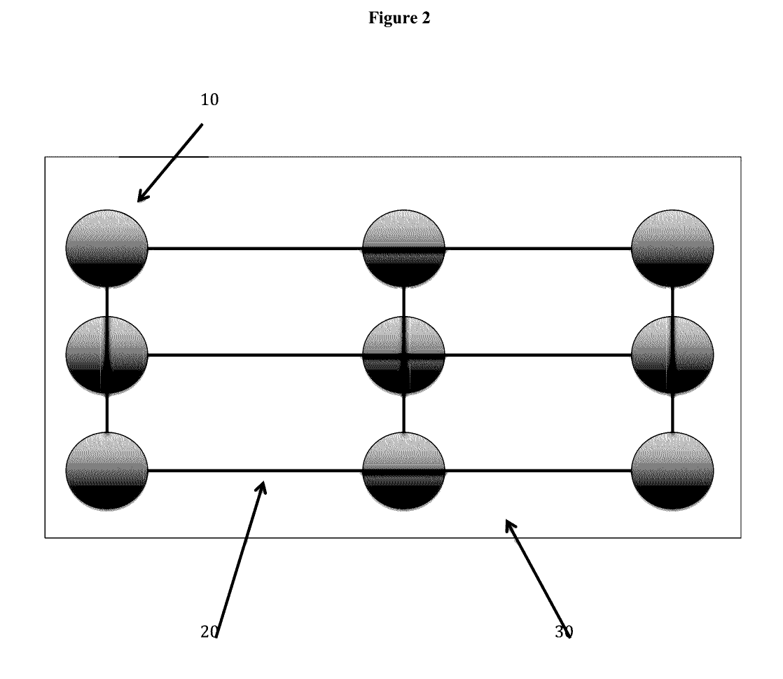

Referring to FIG. 2, this shows a simple 3 by 3 array useful in the present disclosure. It is comprised of wells 10 and channels 20 that interconnect the wells to form a simple mesh network. The array can be fabricated on a number of substrates such as glass, polymer films, molded micro plates, and plates with electrode arrays. The array of the can be a simple n×n with n=1, or where each “n” can range from 1 to about 10,000, e.g., n is 1 to about 100. The well diameter to contain the neurons can range from about 10 microns to about 25.4 millimeters. The well can be in any number of geometric shapes, such as a circle, square, or polygon. The wells 10 can also contain a volume of liquid to nourish the cells and promote growth and generally can contain micro liters to milliliters of appropriate growth media.

The substrate 30 can be fabricated by a number of techniques well known in the art such as liquid casting, injecting molding, thermal and/or UV micro embossing, micro machining, thermoforming, and/or high pressure stamping. In one embodiment, injection molding and/or micro embossing are employed. In one embodiment, the substrate may be transparent if subsequent optical analysis is to be conducted on the topological network. However, if electrophysiology is desired to observe the behavior of the network during exposure to a biologically active compound, the substrate need not be transparent.

The channels 20 that connect the wells 10 can vary in width, length and depth depending on how complex and what type of topological network is being fabricated. In one embodiment, the channels are generally about 2 microns to about 100 microns in width, e.g., about 5 microns to about 15 microns. The channel depth is approximately equal to the depth of the well 10 walls. The channels can be fabricated by micro molding, micro embossing, micro machining, or laser ablation. The channel allows axons that are generated by the neurons to communicate to adjacent or distant wells 10. The wells 10 can be populated with neurons only or other cells types as well such as neuromuscular, cardiac, liver, kidney, pancreas, and skin to create complex neuron organ in vitro models.

In one example using UV micro embossing on a polymer film, the process formed a neuron topological substrate. In this example the wells 10 and channels 20 were created in one step by the use of an embossing tool that creates the microstructure geometries of interest in a high speed roll to roll process. The wells had a diameter of approximately 1 millimeter and the channels that interconnect the wells had a width of about 10 microns.

In another example a 384 well plate well known in the art was converted into a neuron topological substrate by laser engraving or ablating 10 micron channels between the wells to form a mesh topological network on the plate. In yet another example a 96 well electrode plate was converted into a topological neuron network by micro machining 10 micron channels between the wells.

In all the aforementioned examples the topological arrays with the appropriate mammalian cells, growth media and incubation conditions are subsequently exposed (the wells) to active biological compounds. The topological neuron arrays can then be either optically or electrically monitored to observe the physical and logical behavior of the axon cabling between the cells of the mesh network. In one embodiment, a neuron in the array is multipolar. In one embodiment, a neuron in the array is pseudo-unipolar. In one embodiment, a neuron in the array is bipolar. In one embodiment, a neuron in the array is a Purkinje cell. In one embodiment, a neuron in the array is a granule cell. In one embodiment, a neuron in the array is a pyramidal cell. In one embodiment, a neuron in the array is a chandelier cell. In one embodiment, a neuron in the array is a spindle neuron. In one embodiment, a neuron in the array is a stellate cell.

In one embodiment, the wells in the array have the same growth medium. For example, for neurons, an exemplary medium is BrainPhys (Stem Cell Technologies, Vancouver, Canada), and for heart cells, an exemplary medium is medium for iPS cells. In one embodiment, certain wells having neurons, e.g., hippocampal, cortical or dorsal root ganglion neurons, are adjacent to wells having motor neurons and those cells are maintained in the same medium.

In one embodiment, the length of a channel is about 5 microns to about 1 millimeter. In one embodiment, the length of a channel is about 5 microns to about 30 microns. In one embodiment, the length of a channel is about 10 microns to about 20 microns.

In one embodiment, the diameter (width) of a channel is about 5 microns to about 250 microns. In one embodiment, the width of a channel is about 5 microns to about 30 microns. In one embodiment, the width of a channel is about 10 microns to about 20 microns.

In one embodiment, when cells in different wells in the array have different growth medium, the channels that link wells may be of a size that limits media diffusion between adjacent wells. For example, in an array with brain, liver and heart cells, the channels may be of a diameter of about 5 to about 100 microns, e.g., from about 15 to about 25 microns. In one embodiment, the diameter of the channel minimizes diffusion into adjacent wells by the medium due to surface tension.

In one embodiment, to allow for axon generation, the channels are from about 15 to about 25 microns in diameter. In one embodiment, the base of the channel and the base of the well are at the same level. In one embodiment, the channel is U-shaped. In one embodiment, the shape of the channel is a cylinder. In one embodiment, the channel is angular in shape, e.g., a rectangular prism (cuboid), a triangular prism or a hexagonal prism.

The subject matter herein is described by example and different ways of practicing the subject matter have been described. However the subject matter covered by this application is not limited to any one specific embodiment or use or their equivalents. While particular embodiments of the method for fabricating a substrate having cell micro arrays with subsequent drug dosing have been described it will be appreciated by those skilled in the art that changes and modifications may be made thereto without departing from the invention and as set forth in the following claims.

What is claimed is:

1. A biocompatible substrate comprising a network of wells for culturing cells in vitro and channels connecting the wells, comprising:
two or more wells including media and connected by channels configured form a network between the two or more wells and to facilitate intercellular connections, wherein at least one of the wells has neurons, or stem or progenitor cells capable of differentiating into neural cells.

2. The substrate of claim 1 wherein the network is a ring, mesh, star, line, tree or bus topology.

3. The substrate of claim 1 wherein the substrate comprises glass or a synthetic polymer.

4. The substrate of claim 1 wherein the substrate comprises an electrode array.

5. The substrate of claim 1 wherein at least one well includes neuromuscular, cardiac, liver, kidney, pancreas, or skin cells.

6. The substrate of claim 1 wherein at least one well includes motor neurons.

7. The substrate of claim 1 wherein at least one well includes non-motor neurons.

8. The substrate of claim 7 wherein the non-motor neurons comprise cortical neurons, hippocampal neurons or dorsal root neurons.

9. The substrate of claim 1 wherein adjacent wells have different cell types.

10. The substrate of claim 1 wherein at least one channel has a diameter of about 5 to 25 microns.

11. The substrate of claim 1 wherein at least one channel has a length of about 5 to 25 microns.

12. A method of making a biocompatible substrate comprising a network of wells for culturing cells in vitro and channels connecting the wells, comprising:
providing a multi-well plate, wherein the diameter of the wells in the plate is about 10 microns to about 25000 microns; and
fabricating one or more channels between one or more of the wells, wherein the width of the channels is about 2 microns to about 250 microns, thereby forming a network of interconnected wells.

13. The method of claim 12 wherein the channels have a width of about 5 to about 25 microns.

14. The method of claim 12 wherein the channels have a length of about 5 to about 25 microns.

15. The method of claim 12 wherein the network is a ring, mesh, star, line, tree or bus topology.

16. The method of claim 12 which is formed by liquid casting, injecting molding, thermal and/or UV micro embossing, micro machining, thermoforming, and/or high pressure stamping.

17. A method to monitor cellular activity, comprising:
providing the substrate of claim 1;
contacting the cells in the wells with one or more compounds; and
monitoring the activity of the cells after contact.

18. The method of claim 17 wherein electrical activity is monitored.

19. The method of claim 17 wherein the non-motor neurons comprise cortical neurons, hippocampal neurons or dorsal root neurons.

20. The method of claim 17 wherein one or more of the channels comprise axons.
